(19) United States
(12) Patent Application Publication
(10) Pub. No.: US 2024/0353692 A1
(43) Pub. Date: Oct. 24, 2024

(54) Eyewear with Optical Sensor for Selective Filtering and Enhancing of Electromagnetic Radiation having Non-Visual Physiological Responses

(71) Applicant: Timeshifter, Inc., southampton, NY (US)

(72) Inventors: Mickey Beyer-Clausen, Southampton, NY (US); Steven Lockley, Somerville, MA (US); Fredric Maxik, Orlando, FL (US)

(21) Appl. No.: 18/688,238

(22) PCT Filed: Aug. 30, 2022
(86) PCT No.: PCT/US2022/075667

§ 371 Date: Feb. 29, 2024

Related U.S. Application Data
(60) Provisional application No. 63/260,708, filed on Aug. 30, 2021.

(51) Int. Cl. G02C 7/10; A61M 21/00; G01J 3/44

(52) U.S. Cl. CPC G02C 7/101; A61M 21/00; G01J 3/4406; A61M 2021/0044

(57) ABSTRACT

An eyewear device a including a frame an optical sensor to measure environmental light, a lens including a transmission control region that is switchable between first and second configurations, operable to reflect and/or absorb light within the visible spectrum to reduce and/or enhance non-visual responses to light, and operable to permit light within the affected wavelength range to pass therethrough in the second configuration. Electrodes electrically coupled to the transmission control region may enable an electric current to flow through the transmission control region. A control system electrically coupled to the electrodes and the optical sensor may control the operation of the eyewear device.

### FIELD OF THE INVENTION

The present invention relates to eyewear for selective enhancement and filtering of electromagnetic radiation incident upon the eyes of an individual that alters non-visual responses to light.

### BACKGROUND OF THE INVENTION

Eyewear to mitigate the effect of sunlight have primarily been limited to visual responses, namely, reducing the overall intensity of light incident on the eyes of the wearer. Recently, eyewear intended to specifically filter blue light have been employed, particularly for users who are looking at backlight electronic displays, which frequently have enough blue light to effect melatonin production in the body of the wearer. However, such eyewear is constrained to permanent filtering of blue light, and are not operable to be selective in when such blue light is permitted to be observed by the wearer when it may be preferable, for example, during typical waking hours or when the wearer otherwise would not benefit from melatonin production. Additionally, such devices do not have the capability of increasing the blue light above the intensity level present in the environmental light they would observe. Accordingly, there is a need in the art for an eyewear device that can selectively alter the intensity of light, either increasing or decreasing, within a target wavelength range to have an effect on a non-visual physiological response in the wearer.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to an eyewear device comprising a frame, an optical sensor positioned on the frame and configured to measure environmental light and transmit a signal indicated the spectral power distribution of the environmental light, a lens comprising a transmission control region, the transmission control region that is switchable between a first configuration in the absence of an electric current and a second configuration by the application of an electric current, operable to at least one of reflect and absorb light within the visible spectrum to one of reduce or enhance non-visual responses to light, defining an affected wavelength range, and operable to permit light within the affected wavelength range to pass therethrough in the second configuration to one of reduce and enhance non-visual responses. The eyewear device further includes electrodes electrically coupled to the transmission control region and configured to enable an electric current to flow through the transmission control region and a control system electrically coupled to the electrodes and the optical sensor. The control system includes a processor positioned in communication with the optical sensor and configured to receive the signal therefrom, a memory device operatively coupled to the processor, and a wireless communication device operatively coupled to the processor. The wireless communication device is configured to transmit environmental light measurements from the optical sensor and receive transmissions from a remote computerized device indicating a command to at least one of increase exposure to the affected wavelength range and decrease exposure to the affected wavelength range responsive to at least one of the environmental light measurements and a circadian shift protocol. The eyewear further includes a power source electrically coupled to the processor, the memory device, the wireless communication device, and the electrodes. The processor is configured to control the operation of the power source to apply a current to effectuate an electric current between the electrodes responsive to the received command and the signal from the optical sensor.

In some embodiments, the affected wavelength range is defined as from 450 nm to 555 nm from the environment, characterized in that the affected wavelength range is a non-visual response wavelength range. In some embodiments, the non-visual response is at least one of melatonin suppression, alertness, mood alteration, circadian phase resetting, and pupillary constriction.

In some embodiments, the transmission control region is configured to at least one of reflect light having a wavelength outside the affected wavelength range, absorb light having a wavelength outside the affected wavelength range, and convert light having a wavelength within the affected wavelength range into light having a wavelength below the affected wavelength range by performing a Stokes shift. In some embodiments, the transmission control region is configured to at least one of reflect light having a wavelength within the affected wavelength range and absorb light having a wavelength within the affected wavelength range.

In some embodiments, the eyewear device may further comprise conversion material positioned in optical communication with each of the environmental light and the lens and configured to absorb environmental light having a wavelength that is shorter than or greater than the affected wavelength range incident thereupon and emit light within the affected wavelength range. The conversion material may be positioned on an upper portion of the lens. The conversion material may be converted to emit light having a peak wavelength intensity within a range from 450 nm to 555 nm. The conversion material may comprise at least one of a phosphor material, a quantum dot material, and a photosensitive dye. In some further embodiments, the eyewear device may further comprise a conversion material filter configured to be transitioned between first and second configurations, with the first configuration operable to at least one of reflect and absorb light within the affected wavelength range and the second configuration configured to permit light within the affected wavelength range to pass therethrough. The conversion material filter may be configured to be switched between the first and second configurations by application of an electrical current. In such embodiments, the eyewear device may further comprise electrodes electrically coupled to the conversion material filter and configured to enable an electrical current to flow through the conversion material. The wireless communication device may further be configured to receive transmissions from a remote computerized device indicating a command to at least one of increase exposure to the affected wavelength range and decrease exposure to the affected wavelength range, and the processor may be configured to control the operation of the power source to apply a current to effectuate an electric current between the electrodes responsive to the received command.

In some embodiments, the processor may be configured to determine at least one of a melanopic daylight efficacy ratio (DER), an S-cone opic DER, an M-cone opic DER, an L-cone opic DER, and a rhodopic DER of environmental light measurements. In further embodiments, the processor may be configured to control the operation of the power source to apply a current to effectuate an electric current between the electrodes responsive to each of the received command and the at least one DER ratio. In some embodiments, the received command may be to avoid blue-enriched light exposure and the DER determined by the processor is a melanopic DER that is greater than a threshold melanopic DER value. The threshold ratio may be within a range from 0.1 to 0.4. The processor may be configured to operate the power source to apply a current to effectuate an electric current between the electrodes such that a melanopic DER of light emitted by the eyewear is below the threshold ratio.

In some embodiments, the received command may be to increase blue-enriched lift exposure and the DER is a melanopic DER that is less than a threshold ratio. The threshold may be within a range from 0.6 to 0.9. In further embodiments, the processor may be configured to operate the power source to apply a current to effectuate an electric current between the electrodes such that a melanopic DER of light emitted by the eyewear is above the threshold ratio.

Further embodiments of the invention may be directed to a kit comprising a first eyewear that comprises a frame and a lens comprising a transmission control region configured to at least one of reflect and absorb light within the visible spectrum to one of reduce or enhance non-visual physiological responses to light, defining an affected wavelength range. The kit may further comprise a second eyewear that comprises a frame and a lens comprising a transmission control region configured to at least one of permit light within the affected wavelength range to pass therethrough and absorb environmental light having a wavelength that is shorter than or greater than the affected wavelength range incident thereupon and emit light within the affected wavelength range to one of reduce and enhance non-visual physiological responses.

In some embodiments, at least one of the first eyewear and the second eyewear may further comprise an optical sensor positioned on the frame and configured to measure environmental light and transmit a signal indicated the spectral power distribution of the environmental light and a control system electrically coupled to the optical sensor. The control system may comprise a processor positioned in communication with the optical sensor and configured to receive the signal therefrom, a memory device operatively coupled to the processor, and a wireless communication device operatively coupled to the processor and configured to transmit environmental light measurements from the optical sensor.

### BRIEF DESCRIPTION OF THE DRAWINGS

FIG. 1 is a perspective view of eyewear according to an embodiment of the invention.

FIG. 2 is a schematic view of a lens of eyewear according to an embodiment of the invention.

FIG. 3 is a schematic view of a control system according to an embodiment of the invention.

FIG. 4 is a perspective view of a kit comprising multiple eyewear according to an embodiment of the invention.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

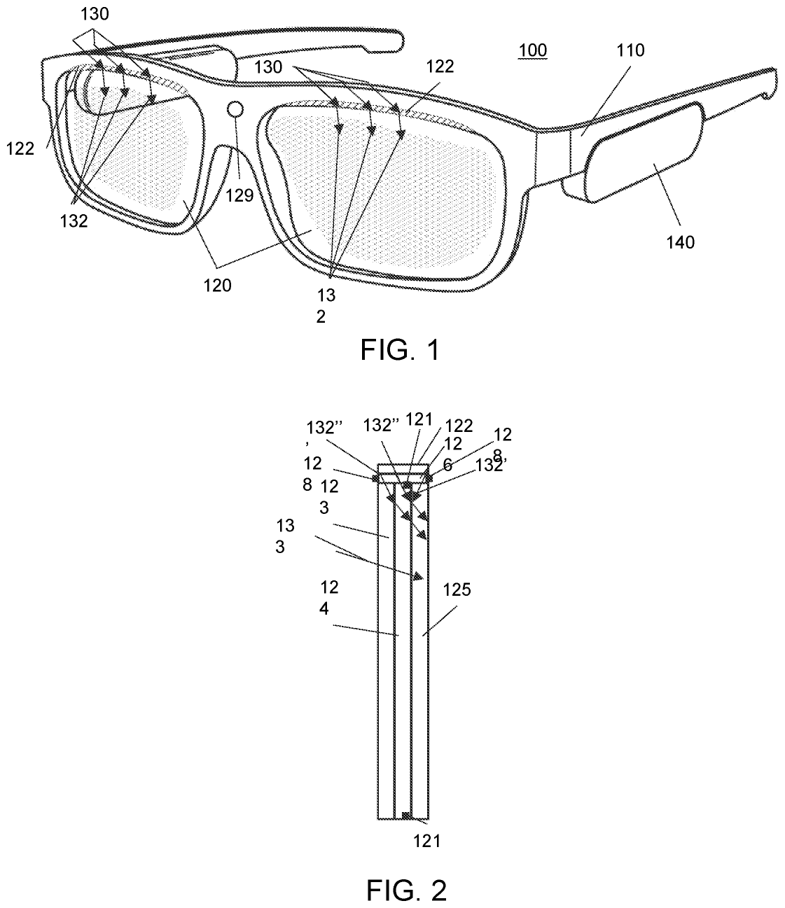

An embodiment of the invention, as shown and described by the various figures and accompanying text, provides a device, in some embodiments in the form of eyeglasses, configured to facilitate circadian shifting by either enhancing or decreasing blue light incident upon a user's eyes. Referring now to FIG. 1, eyewear 100 according to an embodiment of the invention is presented. The eyewear 100 may comprise a frame 110 and one or more lenses 120. The frame 110 may be configured to be worn by an individual such that the lenses 120 are positioned generally adjacent to and in front of the eyes of the individual.

The lenses 120 may comprise one or more conversion materials 122. The conversion materials 122 may be a material that absorbs photons from the environment at a first wavelength, namely an environmental wavelength, or within an environmental wavelength range, and emits photons at a second wavelength, namely a converted wavelength, or within a converted wavelength range that is different from the environmental wavelength. The environmental wavelength may be shorter than or greater than the converted wavelength range. Types of materials that perform this wavelength shift include, but are not limited to, phosphors, quantum dots, photosensitive dyes, and the like. It is contemplated and included within the scope of the invention that any conversion material may be used in the eyewear. In some embodiments, the conversion materials 122 may be positioned along a top or upper portion of the lens. Such positioning may be advantageous to receiving environmental light, particularly sunlight, that tends to be incident on the lens 120 from a generally upward direction.

As shown in FIG. 1, environmental light 130 may be incident upon the conversion materials 122. The environmental light 130 may comprise electromagnetic radiation within a wavelength range that includes the environmental wavelength that the conversion material 122 may be operable to convert. In some embodiments, the conversion material 122 may be configured to perform a Stokes shift and effectively increase the wavelength of the environmental wavelength, emitting electromagnetic radiation 132 at a converted wavelength that is greater than the wavelength than the environmental wavelength. In some embodiments, the conversion material 122 may be configured to perform an anti-Stokes shift and effectively decrease the wavelength of the environmental wavelength, emitting electromagnetic radiation at a converted wavelength that is less than the wavelength of the environmental wavelength.

In order to increase the affect the environmental light has on the circadian system and other non-visual light responses of the individual wearing the eyewear 100, the conversion material 122 may be configured to emit light within a wavelength range that enhances non-visual responses to light, such as, for example increasing melatonin suppression, increasing alertness, increasing circadian rhythm resetting, and increasing pupil constriction, when incident upon the eyes of the individual. Such an emission is exemplary only, and the conversion material 122 may be configured to emit light within any wavelength range having a biological effect, such as a non-visual light response in the wearer. Such a wavelength range includes the range from 450 nanometers (nm) to 500 nm, light that is generally perceived as blue light, this range defining a converted wavelength range. Another wavelength range includes the range from 450 nm to 550 nm. Another wavelength range includes the range from 450 nm to 555 nm. Further embodiments may be configured to emit radiation within a converted wavelength range from 465 nm to 490 nm, and/or having a peak wavelength intensity/being centered from 465 nm to 480 nm. Accordingly, the conversion material 122 may comprise material operable to convert electromagnetic radiation comprised by environmental light outside this range into radiation within this range. In some embodiments, the conversion material 122 may comprise multiple materials, with one material configured to perform a Stokes shift on electromagnetic radiation having a wavelength that is below the converted wavelength range, i.e. below 450 nanometers, including light in the violet and ultraviolet wavelength range, and another material configured to perform an anti-Stokes shift on electromagnetic radiation having a wavelength that is above the converted wavelength range, i.e. above 485 nm. Such embodiments may facilitate thermal management, with the heat generation of Stokes shifts being offset by the cooling effect of performing an anti-Stokes shift. Indeed, the thermal energy produced from the material performing the Stokes shift may be conducted to the material performing the anti-Stokes shift to replenish the necessary thermal energy in the crystal lattice of the material performing the anti-Stokes shift, where applicable.

The conversion material 122 may emit the electromagnetic radiation 132 within the converted wavelength range into the lens 120. In some embodiments, an additive transmission medium may be applied between the conversion material 122 and the lens 120, including, but not limited to, oils, gels, light tubes, and any other substance or structure that may facilitate transmission of radiation therebetween. The lens 120 may be configured to cause the emitted radiation 132 to propagate out of the lens 120 in the direction of the individual wearing the eyewear 100 and not away from the individual. In some embodiments, the lens 120 may comprise a transmission control region 124. The transmission control region 124 may be a portion of or the entirety of the lens 120 and may be configured to control the direction of transmission of electromagnetic radiation within and out of the lens 120. In some embodiments, the transmission control region 124 may be configured to prevent the emitted radiation 132 from transmitting through the lens 122 and outward therefrom away from the individual.

The eyewear 100 may further comprise an optical sensor 129. The optical sensor 129 may be carried by the frame 110 and positioned such that environmental light is incident upon it. The optical sensor 129 may be configured to measure the spectral power distribution (SPD) of the environmental light and transmit a signal to a connected electronic device comprising the SPD of the environmental light. The optical sensor 129 may be any type of device operable to measure environmental light, including, but not limited to, photodiodes, photovoltaics, photoconductive devices, phototransistors, and the like. In the present embodiment, the optical sensor 129 is positioned on a bridge portion of the frame 110. It is contemplated and included within the scope of the invention that the optical sensor 129 may be positioned at any place on the frame 110. Additionally, in some embodiments, the eyewear 100 may comprise a plurality of optical sensors 129 each individually operable to measure the SPD of environmental light, and may be positioned at various positions on the frame 110, which may facilitate a more accurate measurement of environmental light.

Referring now to FIG. 2, the transmission control region 124 is shown positioned between an outer portion 123 of the lens 120 and an inner portion 125 of the lens 120. In some embodiments, the transmission control region 124 may be configured to reflect emitted radiation 132′ that is propagating through the lens 120 in the direction of the outer portion 123 and that would otherwise exit the lens 120 away from the individual. The emitted radiation 132′ may be reflected back into the inner portion 123 in the direction of the individual. In some embodiments, the transmission control region may be configured to selectively reflect radiation within the converted wavelength range while permitting radiation outside that range to pass therethrough. The transmission control region 124 may further be configured to refract light emitted from the conversion material 122 into the transmission control region 124 at the point the emitted radiation 132 enters the transmission control region 124 to avoid distortion of environmental light entering the lens 120 so that the individual wearing the eyewear 100 has difficulty seeing. The transmission control region 124 may further be configured to permit light within the converted wavelength range to propagate therethrough, such that light within the converted wavelength range may enter at the outer portion 123, either as emitted light 132′″ or from the environment 133, pass through the transmission control region 124 and the inner portion 125, and be incident upon the individual.

In some embodiments, the transmission control region 124 may be passive, always performing the above-described behavior of selectively reflecting emitted radiation 132 towards the individual. In some embodiments, the transmission control region 124 may be active and selectively controllable to reflect emitted radiation 132 as described in a first condition and have a different transmission profile in a second condition. For example, in the second condition, the transmission control region 124 may be configured to either absorb light within the converted wavelength range or otherwise prevent or inhibit the transmission of light within the converted wavelength range in the direction of the individual, thereby reflecting or absorbing environmental light 133 and emitted radiation 132″, 132′″ within the converted wavelength range away from the individual and permitting emitted radiation 132′ to pass therethrough towards the outer portion 123 and away from the individual. In another embodiment, in the second condition, the transmission control region may absorb most or all light within the converted wavelength range. The transmission control region 124 may be any type of structure or device that enables such functionality. For example, the transmission control region 124 may be a polymer dispersed liquid crystal (PDLC) that may be opaque to radiation within the converted wavelength range when an electrical current is applied and transparent and/or have a significantly increased transmittance when a current is not applied. Accordingly, the transmission control region 124 may comprise electrodes 121 configured to permit a current to be applied therethrough. As another example, the transmission control region may comprise a ferromagnetic ink configured to be positioned in the first configuration when a magnetic field having a first pole alignment is applied thereto and in the second configuration when a magnetic field having a second pole alignment that is different from the first pole alignment is applied thereto. In such embodiments, one or both of the electrodes 121 may be connected to an electromagnetic device operable to generate magnetic fields having the first and second pole alignments but running current therethrough in opposite directions. The magnetic fields generated by the electromagnet may flow through the transmission control region 124 and shift the ferromagnetic inks comprised thereby between the first and second configurations. In some embodiments, the transmission control region 124 may comprise a transparent organic light-emitting diode (OLED) material. In some embodiments, the electrodes 121 may be formed of transparent material and may be generally transparent.

In some embodiments, the lens 120 may comprise a conversion material filter 126. The conversion material filter 126 may be operable to selectively block emitted radiation 132 from entering the lens 120 and being incident upon the individual. The conversion material filter 126 may be any device operable to selectively filter emitted radiation 132 in a first condition and permit emitted radiation 132 to pass therethrough in a second condition. For example, the conversion material filter 126 may be a PDLC that may be opaque when an electrical current is applied and transparent and/or have a significantly increased transmittance when a current is not applied. Accordingly, the conversion material filter 126 may comprise electrodes 128 configured to permit a current to be applied therethrough. The conversion material filter 126 may be configured to be opaque to a wavelength range including the wavelength of the emitted radiation 126, i.e. the converted wavelength range, or substantially greater, including, either individually or collectively, the visible spectrum, wavelengths within the UV spectrum, and wavelengths within the IR spectrum.

It is contemplated and included within the scope of the invention that the eyewear 100 may be corrective eyewear, addressing vision problems for which eye glasses are typically worn, including, but not limited to, astigmatism, myopia, hypermetropia, presbyopia, and the like. Accordingly, in some embodiments, at least one or both of the outer portion 123 and the inner portion 125 may be refractory. Furthermore, the transmission control region 124 may be configured to avoid interfering with refraction of either of the outer or inner portions 123, 125.

In some embodiments, the eyewear 100 may comprise the transmission control region 124 and not comprise the conversion material 122. In some embodiments the eyewear 100 may comprise the conversion material 122 and not comprise the transmission control region 124. In embodiments where the eyewear 100 comprises only the transmission control region 124, the transmission control region 124 may be passive and configured to one of reflect, absorb, and/or convert environmental light incident thereupon within the converted wavelength range, thereby reducing the non-visual effects of environmental light. In alternative embodiments, the transmission control region 124 may be configured to convert environmental light within a wavelength range outside the converted wavelength range to light within the converted wavelength range, thereby increasing the non-visual effects of environmental light. In some embodiments, the transmission control region may be active and switchable between two configurations by application of an electric current as described above, in a first configuration permitting environmental light within the converted wavelength range to pass therethrough and in a second configuration to one of reflect and absorb environmental light within the converted wavelength range.

While the eyewear 100 is disclosed as being configured in one embodiment to alter the incidence of light within a particular wavelength range that is known to be biologically effective for the particular hormone melatonin, it is contemplated and included within the scope of the invention that the structure of the eyewear 100 may be used in a device that is configured to alter the incidence of environmental light within a wavelength range known to be effective for any other hormone of the human body that is known to be modified by external light exposure, including, but not limited to, leptin, ghrelin and cortisol. Such embodiments may have target wavelength ranges akin to the melatonin suppressing wavelength range, but instead is within a range related to the target hormone.

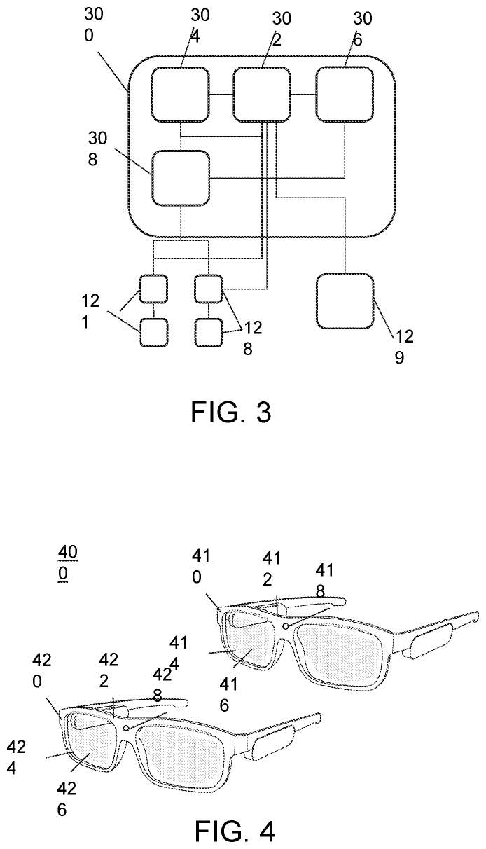

As shown in FIG. 1, the eyewear 100 may comprise a control system 140. The control system 140 may be connected to the electrodes 121, 128 of the transmission control region 124 and the conversion material filter 126 so as to independently control the operation thereof. The control system 140 may be carried on the frame 102 of the eyewear 100, facilitating physical connection to the electrodes 121, 128. Referring now to FIG. 3, a schematic view of a control system 300 according to an embodiment of the invention is presented. The control system 300 may comprise a processor 302, a memory device 304, a wireless communication device 306, a power source 308. The processor 302 may be any type of processing device as is known in the art, including, but not limited to, microprocessors, integrated circuits (IC), field-programmable gate arrays (FPGAs), and the like. The memory device 304 may be operatively coupled to the processor 302 and may be any device operable to store data non-transitively thereon, including, but not limited to, hard disk drives, solid-state drives, flash drives, secure digital (SD) cards, PROM, EPROM, EEPROM, and any other non-volatile memory device. The wireless communication device 306 may be any device capable of wireless communication utilizing wireless transmission standards as are known in the art, including, but not limited to, 802.xx standards, such as WiFi, Bluetooth, Zigbee, and Z-wave, LiFi, cellular communication including CDMA, TDMA, 3G, 4G, and 5G standards, and the like. Moreover, the wireless communication device 306 may be operable to communicate across a network, including Personal Area Networks (PANs), Local Area Networks (LANs), Wide Area Networks (WANs) such as the Internet, as well as communicate directly with other computerized devices. The wireless communication device 306 may be positioned in communication with the processor 302 and operable to transmit and receive commands to and from the processor 302. The power source 308 may be connected to and configured to provide power to each of the processor 302, the memory device 304, and the wireless communication device 306. The power source 308 may be any device capable of delivering such power, including, but not limited to, batteries, capacitors, including super capacitors, and the like. Moreover, the power source 308 may include charging and voltage-changing and regulating circuitry as is necessary to provide electricity of varying voltages to the various components of the eyewear and to facilitate recharging of the power source 308, including charging by physical connection and by wireless charging, such as induction charging.

The processor 302 may be configured to execute software stored on the memory device 304 that enables control of the transmission control region 124 and the conversion material filter 126 as described above, namely, changing the respective structures between first and second conditions that changes the transmission and reflection characteristics thereof. Specifically, the processor 302 may be operatively coupled to the electrodes 121, 128 and/or the power source 308, and any other necessary control circuitry, such as relays, to control the flow of current to electrodes 121, 128 to selectively control the changing between the first and second conditions of the transmission control region 124 and the conversion material filter 126.

As mentioned above, the wireless communication device 306 may be configured to communicate wirelessly with a remote computerized device. An example of such communication is provided in U.S. patent application Ser. No. 16/432,544 titled Method and System for Generating and Providing Notifications for a Circadian Shift Protocol, the content of which is incorporated herein by reference. In the '544 application, a circadian shift protocol is determined to effectuate a change in the circadian rhythm of a user. Part of the protocol includes seeking exposure to and avoiding exposure to blue light at different times of day, with indications to seek or avoid blue light being provided to the user.

In the present embodiment, a remote computerized device may transmit a command to the processor 302 via the wireless communication device 306 to operate one or both of the transmission control region 124 and the conversion material filter 126 to selectively increase or decrease the exposure of the individual to light within the converted wavelength range. Specifically, when a command to increase exposure is received, the processor 302 may operate the transmission control region 124 in its first configuration to reflect and otherwise permit light within the converted wavelength range to be incident upon the user and operate the conversion material filter 126 in its second condition to permit emitted radiation 132 from the conversion material 122 to pass therethrough so as to be incident upon the individual. Conversely, when a command to decrease exposure is received, the processor 302 may operate the transmission control region 124 in its second condition to reduce light within the converted wavelength range from being incident upon the individual and operate the conversion material filter 126 in its first condition to prevent the transmission of emitted radiation 132 therethrough. In some embodiments, a plurality of commands may be received and stored in the memory device 304 for subsequent performance. For example, when a protocol is developed as indicated in the '544 application, all elements of that protocol related to seeking out and avoiding blue light may be transmitted to the wireless communication device 306 and stored in the memory device 304, and the processor 302 may access and execute those commands in accordance with the time to seek out or avoid bright or blue light as indicated by the protocol, selectively increasing or decreasing the amount of light containing the converted wavelength range the individual experiences through control of the conditions of the transmission control region 124 and the conversion material filter 126.

Additionally, the processors 302 may be coupled to the optical sensor 129 and configured to receive signals transmitted therefrom regarding the SPD of environmental light. The processor 302 may be configured to control the operation of the power source 306 to operate one or both of the transmission control region 124 and the conversion material filter 126 to selectively increase or decrease the exposure of the individual to light within the converted wavelength range responsive to one or both of the signal received from the optical sensor 129 and a command received from a remote computerized device, as described above. For example, the processor 302 may receive a command to decrease the amount of light within the converted wavelength range the user is exposed to, and the signal received from the optical sensor 129 may indicate significant levels of light within the converted wavelength range in the SPD of the environmental light. In such a scenario, the processor 302 may be configured to the transmission control region 124 in its second condition to reduce light within the converted wavelength range from being incident upon the individual and operate the conversion material filter 126 in its first condition to prevent the transmission of emitted radiation 132 therethrough. As another example, when a command to increase exposure to light within the converted wavelength range is received, and the SPD signal from the optical sensor 129 indicates an inadequate intensity of light within the converted wavelength range in the environment, the processor 302 may operate the transmission control region 124 in its first configuration to reflect and otherwise permit light within the converted wavelength range to be incident upon the user and operate the conversion material filter 126 in its second condition to permit emitted radiation 132 from the conversion material 122 to pass therethrough so as to be incident upon the individual.

In some embodiments, it is contemplated and included within the scope of the invention that instead of comprising an optical sensor 129, such a sensor may instead be in communication with at least one of the communication device 306 and a remote computerized device in communication with the communication device 306, and that the environmental SPD may be received by the processor 302 via the communication device.

The processor 302 may be configured to determine one or more physiological effects the SPD of environmental light may have on the user. For example, the processor 302 may be configured to determine one or more of a melanopic daylight efficacy ratio (DER), an S-cone-opic DER, an M-cone-opic DER, an L-cone-opic DER, and a rhodopic DER of the SPD of the environmental light. Each of these DERs are known in the art as the efficacy of light on each of the photoreceptors of the human eye. Each may have an independent circadian effect on the observer. The processor 302 may further be configured to, upon determining one or more of the DERs, compare the DER to a threshold value for that DER to determine whether to take an action. Such an action would only be taken only if a command for such an action has been received by the processor 302. For example, the processor 302 may receive a command to avoid light within the converted wavelength range to reduce non-visual responses and may compute a melanopic DER value for the environmental light that is greater than a melanopic DER threshold value. The melanopic DER threshold value may be stored on the memory 304 and may be within a range from 0.2 to 0.5 in this example. Where the melanopic DER value determined by the processor 302 is greater than the melanopic DER threshold value, the processor 304 may be configured to operate the transmission control region 124 in its second condition to reduce light within the converted wavelength range, that in this embodiment may coincide with a melatonin-suppressing wavelength range, from being incident upon the individual and operate the conversion material filter 126 in its first condition to prevent the transmission of emitted radiation 132 therethrough. Furthermore, the DER threshold value utilied by the processor 302 may depend on the command received thereby. As another example, the processor 302 may receive a command to seek exposure to light within the wavelength range to enhance non-visual responses and may measure a melanopic DER value for environmental light that is less than a melanopic DER threshold value. The melanopic DER threshold value may be stored on the memory 304 and may be within a range from 0.7 to 1.2. Where the melanopic DER value determined by the processor 302 is greater than the melanopic DER threshold value, the processor 304 may be configured to operate the transmission control region 124 in its first configuration to reflect and otherwise permit light within the converted wavelength range to be incident upon the user and operate the conversion material filter 126 in its second condition to permit emitted radiation 132 from the conversion material 122 to pass therethrough so as to be incident upon the individual. In some embodiments the melanopic DER threshold value be within a range from 0.2 to 1.2. In some embodiments the melanopic DER threshold value may be within a range from 0.5 to 1.2. In some embodiments, the threshold value may be within a range from 0.1 to 0.4, such that if the melanopic DER value is determined to be less than a threshold melanopic DER value within the range from 0.1 to 0.4, the processor 304 may be configured to operate the transmission control region 124 such that light emitted thereby has a melanopic DER that is less than the threshold melanopic DER value. In some embodiments, the threshold value may be within a range from 0.6 and 0.9, such that if the melanopic DER value is determined to be less than a threshold melanopic DER value within the range from 0.6 to 0.9, the processor 304 may be configured to operate the transmission control region 124 such that light emitted thereby has a melanopic DER that is greater than the threshold melanopic DER value.

The above examples of receiving commands and calculating DERs above/below relevant threshold DER values are exemplary only and non-limiting. It is contemplated and included within the scope of the invention that the processor 302 may receive a command that may determine whether the user should be exposed to or avoid exposure to light within a biologically effective wavelength range that is associated with any one of the photoreceptors associated with each DER, or in any combination, that a threshold DER value for each DER, or the combined DER values, may be stored on the memory 304, and that the processor 302 may operate each of the transmission control region and/or the conversion material filter 126 responsive to the command received by the processor 302 and the measured DER value. As examples, combinations of S-cone and melanopic DERs, L-cone and melanopic DERs, M-cone and melanopic DERs, rhodopic and melanopic DERs, and any other combinations thereof.

References are made above the to the converted wavelength range. It is contemplated that that wavelength range may also be referred to as an affected wavelength range, referencing a wavelength range having a non-visual biological effect on the observer. One example of such a biological effect is melatonin suppression, having a melatonin suppression wavelength range, with light within that wavelength range having the effect of suppressing melatonin production in an individual upon which the light is incident. The physiological response of melatonin suppression is exemplary only and the invention is not limited to this non-visual physiological response. Other types of responses include, but are not limited to, alertness, mood alteration, circadian phase adjustment/resetting, and pupillary constriction.

Referring now to FIG. 4, a kit 400 according to an embodiment of the invention is presented. The kit 400 may comprise at least two eyewear 410, 420. The first eyewear 410 may comprise a frame 412 and at least one lens 414 comprising a transmission control region 416. The transmission control region 416 may be configured to at least one of reflect and absorb light within the visible spectrum to one of reduce or enhance non-visual responses to light, defining an affected wavelength range. The non-visual response may be any non-visual physiological response as is known in the art and as mentioned above. The transmission control region 416 may be at least one of a filter configured to prevent the propagation of light within the affected wavelength range therethrough, thereby reducing the intensity of light within the affected wavelength range observed by a wearer of the eyewear 410.

The second eyewear 420 may similarly comprise a frame 422 and at least one lens 424 comprising a transmission control region 426. The transmission control region 426 may be configured to at least one of permit light within the affected wavelength range to pass therethrough and absorb environmental light having a wavelength that is shorter than or greater than the affected wavelength range incident thereupon and emit light within the affected wavelength range to one of reduce and enhance non-visual physiological responses. For example, the transmission control region 426 may simply be devoid of any substance, material, or characteristic that would prevent, preclude, or inhibit propagation of light within the affected wavelength range from propagating therethrough. As another example, the transmission control region 426 may comprise a substance or material configured to absorb light having a wavelength greater than or less than the affected wavelength range and emit light within the affected wavelength range. Types of materials operable to perform such a wavelength shift are known in the art and described hereinabove.

It is contemplated that the kit 400 may be utilized in conjunction with a protocol configured to effectuate a change in the physiological condition of the wearer. Such a protocol may comprise providing an indication to the user to alternatively reduce or increase exposure to light within the affected wavelength range. When the user receives an indication to avoid exposure to the affected wavelength range, they may don the first eyewear 410. Alternatively, where the user receives an indication to increase exposure to the affected wavelength range, they may don the second eyewear 420.

It is contemplated and included within the scope of the invention that the kit 400 may further comprise a housing (not shown), such as a box or container, within which the eyewear 410, 420 may be contained.

Additionally, one or both of the eyewear 410, 420 may further comprise an optical sensor 418, 428. The optical sensor 418, 428 may be the same as the optical sensors described above, namely, operable to measure the spectral power distribution of environmental light and provide those measurements to a processor. Accordingly, the eyewear 410, 420 that comprises an optical sensor 418, 428 may further comprise a processor, a memory, a wireless communication device, and a power source as described hereinabove, such that the spectral power distribution measured by the optical sensor 418, 428 may be analyzed and/or transmitted to a remote computerized device, such as a device operable to analyze the spectral information, determine how it would affect a protocol associated with the user, determine if the protocol would benefit from or requires use of one of the eyewear 410, 420, and provide an indication to the user to don one of the eyewear 410, 420.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the description of the invention. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

1. An eyewear device comprising:
a frame;
an optical sensor positioned on the frame and configured to measure environmental light and transmit a signal indicating a spectral power distribution of the environmental light;
a lens comprising a transmission control region, the transmission control region being:
switchable between a first configuration in the absence of an electric current and a second configuration by application of an electric current;
operable to at least one of reflect and absorb light within the visible spectrum to one of reduce or enhance non-visual responses to light, defining an affected wavelength range; and
operable to permit light within the affected wavelength range to pass therethrough in the second configuration to one of reduce and enhance non-visual responses;
electrodes electrically coupled to the transmission control region and configured to enable an electric current to flow through the transmission control region; and
a control system electrically coupled to the electrodes and the optical sensor, the control system comprising:
a processor positioned in communication with the optical sensor and configured to receive the signal therefrom;
a memory device operatively coupled to the processor; and
a wireless communication device operatively coupled to the processor and configured to:
transmit environmental light measurements from the optical sensor; and
receive transmissions from a remote computerized device indicating a command to at least one of increase exposure to the affected wavelength range and decrease exposure to the affected wavelength range responsive to at least one of the environmental light measurements and a circadian shift protocol; and
a power source electrically coupled to the processor, the memory device, the wireless communication device, and the electrodes;
wherein the processor is configured to control the operation of the power source to apply a current to effectuate an electric current between the electrodes responsive to the received command and the signal from the optical sensor.

2. The eyewear device of claim 1 wherein the affected wavelength range is defined as from 450 nm to 555 nm from the environment, characterized in that the affected wavelength range is a non-visual response wavelength range.

3. The eyewear device of claim 1 wherein the non-visual response is at least one of melatonin suppression, alertness, mood alteration, circadian phase resetting, and pupillary constriction.

4. The eyewear device of claim 1 wherein the transmission control region is configured to at least one of reflect light having a wavelength outside the affected wavelength range, absorb light having a wavelength outside the affected wavelength range, and convert light having a wavelength within the affected wavelength range into light having a wavelength below the affected wavelength range by performing a Stokes shift.

5. The eyewear of claim 1 wherein the transmission control region is configured to at least one of reflect light having a wavelength within the affected wavelength range and absorb light having a wavelength within the affected wavelength range.

6. The eyewear of claim 1 further comprising conversion material positioned in optical communication with each of the environmental light and the lens and configured to absorb environmental light having a wavelength that is shorter than or greater than the affected wavelength range that is incident thereupon and emit light within the affected wavelength range.

7. The eyewear of claim 6 wherein the conversion material is positioned on an upper portion of the lens.

8. The eyewear of claim 6 wherein the conversion material is configured to emit light having a peak wavelength intensity within a range from 450 nm to 555 nm.

9. The eyewear of claim 8 wherein the conversion material comprises at least one of a phosphor material, a quantum dot material, and a photosensitive dye.

10. The eyewear of claim 9 further comprising a conversion material filter configured to be transitioned between first and second configurations, with the first configuration operable to at least one of reflect and absorb light within the affected wavelength range and the second configuration configured to permit light within the affected wavelength range to pass therethrough.

11. The eyewear of claim 10 wherein the conversion material filter is configured to be switched between the first and second configurations by application of an electrical current, the eyewear further comprising:
electrodes electrically coupled to the conversion material filter and configured to enable an electrical current to flow through the conversion material; and
wherein the wireless communication device is configured to receive transmissions from a remote computerized device indicating a command to at least one of increase exposure to the affected wavelength range and decrease exposure to the affected wavelength range; and
wherein the processor is configured to control the operation of the power source to apply a current to effectuate an electric current between the electrodes responsive to the received command.

12. The eyewear of claim 1 wherein the processor is configured to determine at least one DER ratio from the group of DER ratios comprising a melanopic daylight efficacy ratio (DER), an S-cone opic DER, an M-cone opic DER, an L-cone opic DER, and a rhodopic DER of environmental light measurements.

13. The eyewear of claim 12 wherein the processor is configured to control the operation of the power source to apply a current to effectuate an electric current between the electrodes responsive to each of the received command and the at least one DER ratio.

14. The eyewear of claim 13 wherein the received command is to avoid blue-enriched light exposure and the DER determined by the processor is a melanopic DER that is greater than a threshold melanopic DER value.

15. The eyewear of claim 14 wherein the threshold ratio is within a range from 0.1 to 0.4.

16. The eyewear of claim 15 wherein the processor is configured to operate the power source to apply a current to effectuate an electric current between the electrodes such that a melanopic DER of light emitted by the eyewear is below the threshold ratio.

17. The eyewear of claim 13 wherein the received command is to increase blue-enriched lift exposure and the DER is a melanopic DER that is less than a threshold ratio.

18. The eyewear of claim 17 wherein the threshold ratio is within a range from 0.6 to 0.9.

19. The eyewear of claim 18 wherein the processor is configured to operate the power source to apply a current to effectuate an electric current between the electrodes such that a melanopic DER of light emitted by the eyewear is above the threshold ratio.

20. A kit comprising:
a first eyewear comprising:
a frame; and
a lens comprising a transmission control region configured to at least one of reflect and absorb light within the visible spectrum to one of reduce or enhance non-visual physiological responses to light, defining an affected wavelength range; and
a second eyewear comprising:
a frame; and
a lens comprising a transmission control region configured to at least one of permit light within the affected wavelength range to pass therethrough and absorb environmental light having a wavelength that is shorter than or greater than the affected wavelength range that is incident thereupon and emit light within the affected wavelength range to one of reduce and enhance non-visual physiological responses.

21. The kit of claim 20 wherein at least one of the first eyewear and the second eyewear further comprises
an optical sensor positioned on the frame and configured to measure environmental light and transmit a signal indicating a spectral power distribution of the environmental light; and
a control system electrically coupled to the optical sensor, the control system comprising:
a processor positioned in communication with the optical sensor and configured to receive the signal therefrom;
a memory device operatively coupled to the processor; and
a wireless communication device operatively coupled to the processor
and configured to transmit environmental light measurements from the optical sensor.
